(19) United States
(12) Patent Application Publication
(10) Pub. No.: US 2008/0015543 A1
(43) Pub. Date: Jan. 17, 2008

(54) Cold gas spray for stopping nosebleeds

(76) Inventor: Peng Wang, Naperville, IL (US)

Correspondence Address: THE LAW OFFICE OF RANDALL T. ERICKSON, P.C., 1749 S. NAPERVILLE ROAD, SUITE 202, WHEATON, IL, 60187, US

(21) Appl. No.: 11/821,680

(22) Filed: Jun. 25, 2007

Related U.S. Application Data
(60) Provisional application No. 60/816,082, filed on Jun. 23, 2006.

(51) Int. Cl. A61M 31/00

(52) U.S. Cl. 604/514; 604/73

(57) ABSTRACT

A method and apparatus to treat nosebleeds includes the steps of producing cold air using the input of air into a cooling apparatus and administering the cold air to the inside of the nose. The apparatus includes a cooling device with no moving parts. Preferably the cooling device is a vortex tube or a Peltier-type thermoelectric cooler. The cold air can also be mixed with water to moisturize the cold air.

### TECHNICAL FIELD OF THE INVENTION

The present invention relates to the treatment of nosebleeds, in particular, treatments involving the use of cold temperature.

### BACKGROUND OF THE INVENTION

Nosebleeds are common because the nasal membrane contains many tiny superficial blood vessels that are very fragile. Common causes of nosebleeds include dry air, colds, allergies, sinusitis, physical injury, and drug side effects. Children, in particular, are vulnerable to nosebleeds.

Various remedies for nosebleeds exist, and the idea of using cold temperature to stop nosebleeds is not new. Cold temperature constricts the blood vessels and also increases the blood viscosity. Many medical sources encourage the application of an icepack on the nose when nosebleeds occur. At home, cold, wet towels are often used. Unfortunately, those methods only cool the outside of the nose and have only a slight effect on the inner nasal membrane, where the blood vessels are located. Commercial icepacks also have a tendency to heat up with time. For this reason, these methods cannot stop a nosebleed as quickly as desired.

The present inventor has recognized the need for a method and apparatus that is effective at stopping nosebleeds quickly.

The present inventor has recognized the need for a method and apparatus to treat nosebleeds that is portable, inexpensive, easy to use, quick to take effect, and safe.

### SUMMARY OF THE INVENTION

The present invention provides a method and apparatus to treat nosebleeds that comprises the application of a cold fluid to be sprayed or injected inside the affected nostril of the nose.

According to the preferred embodiment, the cold fluid is air and the apparatus of the present invention comprises an air tank filled with pressurized air connected by a hose to a vortex tube.

The Vortex tube was invented in 1930 by French physicist Georges J. Ranque and later improved by the German physicist Rudolf Hilsch. A vortex tube separates pressurized air into two streams of hot and cold air due to its internal configuration. A description of the configuration and operation of vortex tubes are disclosed in U.S. Pat. Nos. 1,952,281; 4,240,261; and 5,327,728, all herein incorporated by reference. A vortex tube includes an inlet opening for receiving air from a pressurized air tank, a cold air outlet and a hot air outlet.

The cold air outlet of the vortex tube of the present invention is connected to a hose with a nozzle adapted to administer the cold air into the affected nostril of the nose. The warm air outlet discharges to atmosphere. Although in this embodiment the vortex tube inlet opening is connected to an air tank to provide the source of pressurized air, the vortex tube could instead be connected to an air compressor to provide the source of pressurized air.

Because vortex tubes have lower efficiency than traditional air conditioning equipment, the vortex tube has not gained widespread use for cooling. Currently, it is only used for certain industrial spot cooling applications. However, the present inventor has recognized that for nosebleeds, efficiency is not an issue because only a small amount of cooling is needed. Far more important qualities are portability, convenience, safety, cost, and speed of delivering the cold air.

The preferred embodiment apparatus has very few moving parts and is durable and easily transportable. It also produces cold air faster than a conventional air conditioning or refrigerating system. Furthermore, the preferred embodiment apparatus uses breathable air instead of a refrigerant, increasing safety in operation. In addition, the apparatus is particularly effective because the fluid that is applied can reach deep into the nose to the bleeding area to take effect, even when the nose is filled with blood clots or mucus. Moreover, a gas is easy to use, even by children, because it is unnecessary to find the bleeding spot or clean the nose first. In effect, the cold gas finds the bleeding spot as if ice could be applied directly to the bleeding spot, with no mess or undue effort.

This apparatus could also have an attached moisturizer that adds water to the fluid spray, and an attached air pump to replenish the compressed air.

A second embodiment method and apparatus includes a pre-cooled air tank to administer the cold fluid to the user's nose.

Another embodiment method and apparatus for administering a fluid to the user's nose to stop a nosebleed has an aerosol-like refrigerant spray of a safe gas, so that, upon expansion, a cold gas is generated and can be applied inside the user's nose.

Yet another embodiment method and apparatus for administering cold fluid to the user's nose comprises a conventional refrigeration system providing cold air to be applied to the nose. The apparatus could include a small, portable refrigerator for cooling air, or one with a hose attached directly to a cold air output from the evaporator coil.

According to another embodiment method and apparatus for administering a fluid to the user's nose, a supply of gas is cooled as it passes through a thin tube or coil that is cooled from the outside. For example, a pressurized air supply from a tank or from a compressor, pump or fan can supply air through a coil which is cooled by an external supply of ice, cold water, or a spray of gas or liquid applied onto the coil.

According to another embodiment a Peltier-type cooling device can be used to cool air that is applied inside the nostril to stop a nosebleed.

Numerous other advantages and features of the present invention will become readily apparent from the following detailed description of the invention and the embodiments thereof, and from the accompanying drawings.

### BRIEF DESCRIPTION OF THE DRAWINGS

FIG. 1 is a schematic view of the present invention in its preferred embodiment;

FIG. 2 is a schematic view of a second embodiment of the present invention;

FIG. 3 is a schematic view of a third embodiment of the present invention;

FIG. 4 is a schematic view of a fourth embodiment of the present invention;

FIG. 5 is schematic view of a fifth embodiment of the present invention;

FIG. 6 is a schematic view of a sixth embodiment of the present invention;

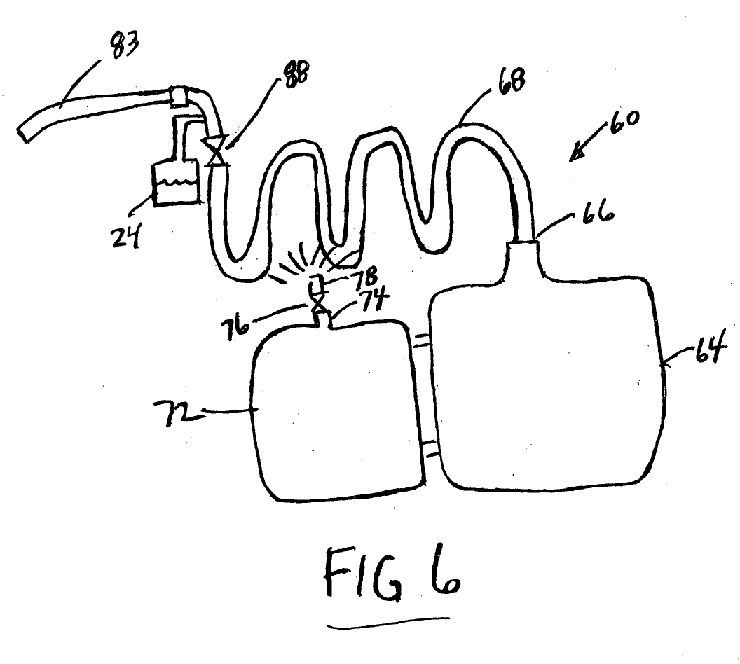

FIG. 7 is a schematic view of a seventh embodiment of the present invention.

### DETAILED DESCRIPTION OF THE PREFERRED EMBODIMENTS

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

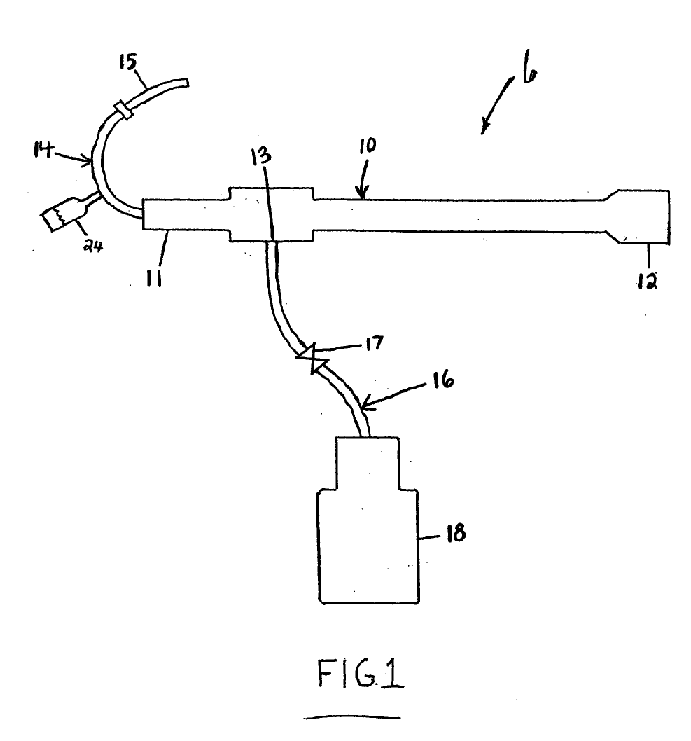

The preferred embodiment apparatus 6 is shown in FIG. 1, and includes a vortex tube 10 having a cold air output 1 1 and a hot air output 12. The cold air output 11 is connected to an output hose 14. Output hose 14 has an applicator or nozzle 15 attached at the end for injecting the cold air into the affected nostril. The nozzle is preferably removable for cleaning or is disposable, for hygienic reasons. Hose 14 may also have an attached moisture reservoir 24. The reservoir 24 can have a supply of water that is delivered as droplets or vapor into the hose 14 by the velocity of air passing through the hose 14 or through a venturi arranged within the hose. Vortex tube 10 has an input 13 which receives air from input hose 16. Input hose 16 is attached to pressurized air supply 18 and can include a valve 17 for controlling the rate of air flow and acting as a shut off.

The hot air outlet 12 can be provided with a vented cover or shroud to prevent hot air from contacting a user. It is also desirable to prevent blood from entering the vortex tube. A bend can be provided in the applicator 15 in a way that the vortex tube will be at a higher position than the bend, wherein gravitational force will prevent blood from flowing into the vortex tube.

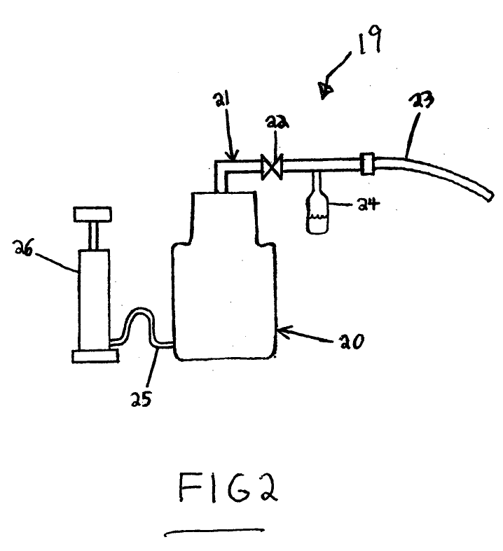

A second apparatus 19 is shown in FIG. 2 and includes a pre-cooled tank 20 filled with pressurized air. Such a tank could be pre-cooled in a freezer and insulated to retain a cold temperature for an extended period of time after removal from the freezer. A hose 21 with a valve 22 is attached to the pre-cooled air tank 20. A nozzle 23 is attached at the end of hose 21 for injecting the cooled air into the affected nostril. The nozzle is preferably removable for cleaning or is disposable, for hygienic reasons. Hose 21 may also have an attached moisture reservoir 24. The reservoir 24 can have a supply of water that is delivered into the hose 21 by the velocity of air passing through the hose 21. Pre-cooled air tank 20 may have a charging hose 25 attaching pre-cooled air tank 20 to an output of an air pump 26.

The air pump 26 could also be used with any of the embodiments of FIGS. 1-5 that use an air tank.

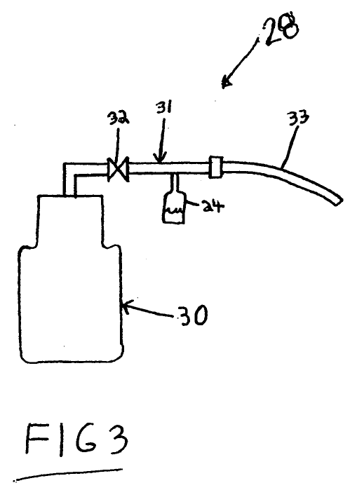

A third embodiment apparatus 28 is shown in FIG. 3 and includes a tank of compressed refrigerant gas 30. Attached to the refrigerant gas tank 30 is a hose 31 with a valve 32 and a nozzle 33 at the end for injecting the cooled refrigerant gas into the affected nostril. The refrigerant gas must be safe for inhalation. The refrigerant gas is selected such that it cools to a great extent when it discharges and expands from the nozzle 33. Alternately, the reservoir can contain a liquid or gas that is already at a low temperature. A mechanism can be provided to prevent adverse over-cooling by a very cold gas to prevent frostbite. The nozzle is preferably removable for cleaning or is disposable, for hygienic reasons. Hose 31 may also have an attached moisture reservoir 24. The reservoir 24 can have a supply of water that is delivered into the hose 31 by the velocity of air passing through the hose 31 or through a venturi within the hose.

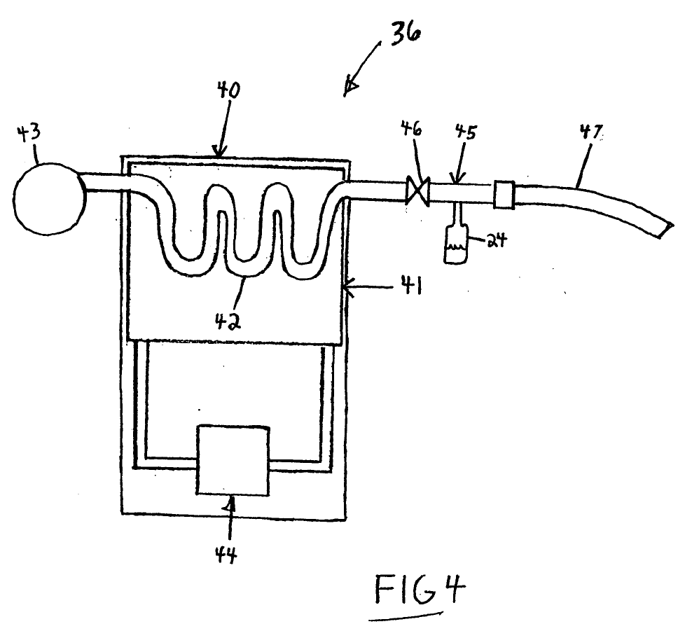

A fourth embodiment apparatus 36 is shown in FIG. 4 that includes a refrigerator 40 comprising an evaporator 41, an air coil 42, a pump or fan 43, and the remaining components of a conventional refrigeration circuit 44, i. e., a circuit that includes a compressor, a condenser, a valve and the evaporator 41. The air coil 42 is connected to hose 45, which includes a valve 46 and a nozzle 47 for injecting the cooled air into the affected nostril. The nozzle is preferably removable for cleaning or is disposable, for hygienic reasons. Hose 45 may also have an attached moisture reservoir 24. The reservoir 24 can have a supply of water that is delivered into the hose 45 by the velocity of air passing through the hose 45 or through a venturi within the hose. Air delivered though the coil 42 is cooled by the evaporating refrigerant. Alternately, the pump or fan 43 could be replaced by a pressurized air tank.

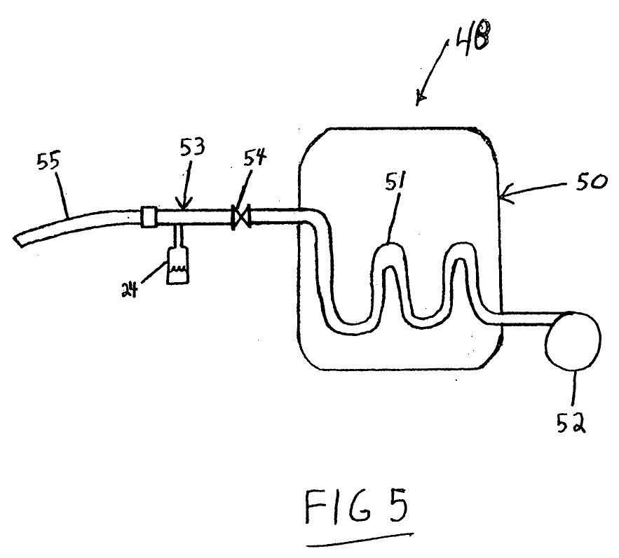

A fifth embodiment apparatus 48 is shown in FIG. 5 and includes a container of cold fluid 50. Container 50 includes air coils 51 inside, and a pump or fan 52 blowing air through air coil 51. The air coil is connected to a hose 53, which can include a valve 54 and a nozzle 55. The nozzle is preferably removable for cleaning or is disposable, for hygienic reasons. Hose 53 may also have an attached moisture reservoir 24. The reservoir 24 can have a supply of water that is delivered into the hose 53 by the velocity of air passing through the hose 53 or through a venturi within the hose. Alternately, the pump or fan 52 could be replaced by a pressurized air tank.

A sixth embodiment apparatus 60 utilizes an air tank 64 of pressurized air having an outlet 66 connected to a tube 68 in the form of a coil. A compressed refrigerant tank 72 is mounted with the air tank 64 and has an outlet 74 connected to a valve 76 that is connected to a nozzle 78 that directs discharged and expanded refrigerant, such as CO₂, at and over the coil. The refrigerant gas cools to a great extent when it discharges and expands from the nozzle 78 and it cools the air passing through the tube 68. The tube 68 is connected to a nozzle 83 at the end thereof for injecting the cooled air into the affected nostril. A valve 88 can be located along the tube 68. The nozzle 83 is preferably removable for cleaning or disposable, for hygienic reasons. The tube 68 can have an attached moisture reservoir 24. The reservoir 24 can have a supply of water that is delivered into the tube 68 by the velocity of air passing through the tube or through a venturi within the tube.

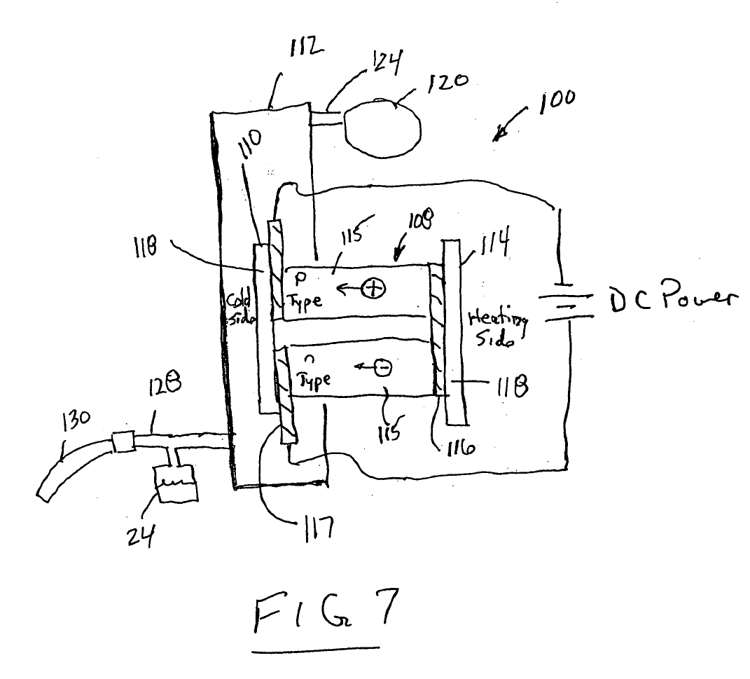

FIG. 7 illustrates another embodiment of the invention wherein a Peltier-type thermoelectric cooling device is used to cool air for application into the nose to stop a nosebleed. A Peltier-type device is a solid state active heat pump which transfers heat from one side of the device to the other side of the device against the temperature gradient using consumption of electrical energy. A Peltier-type device is connected to a DC voltage and one side cools and the other side heats. A Peltier-type cooling device is described in detail in U.S. Pat. No. 6,560,968, herein incorporated by reference.

A seventh embodiment apparatus 100 includes a Peltier-type device 108 that includes a first heat transfer surface 110 on a cooling side within an air chamber 112, and a second heat transfer surface 114 on a heating side outside of the chamber 112. The second heat transfer surface 114 should be protected with a shield to prevent accidental contact by a user.

The device 108 includes thermoelectric elements 115 of semiconductor doped with N-type impurity ions or P-type impurity ions, electrodes 116, 117 of copper or the like, and a ceramic substrate 118 or the like enclosing the electrodes 116, 117. The heat transfer surfaces 110, 114 are provided on the substrate 118.

An air pump or fan 120, or alternately a pressurized air tank, blows air through the chamber 112 via an inlet hose 124 where it is cooled. An outlet hose 128 is connected to an outlet of the chamber and to an applicator or nozzle 130 for application of the cooled air into the user's affected nostril to stop a nosebleed. A valve 138 can be located along the tube 128. The nozzle 130 is preferably removable for cleaning, or is disposable, for hygienic reasons. The hose 128 can have an attached moisture reservoir 24. The reservoir 24 can have a supply of water that is delivered into the tube 128 by the velocity of air passing through the tube or through a venturi within the tube.

From the foregoing, it will be observed that numerous variations and modifications may be effected without departing from the spirit and scope of the invention. It is to be understood that no limitation with respect to the specific apparatus illustrated herein is intended or should be inferred.

The invention claimed is:

1. A method to treat nosebleeds comprising the step of administering cold fluid to the inside of the nose.

2. The method according to claim 1, wherein said step of administering cold fluid is further defined in that said cold fluid is cold air, and said method comprises the further step of producing the cold air using the input of air into a cooling apparatus.

3. The method according to claim 2, wherein said step of producing the cold air is further defined in that said apparatus comprises a cooling device with no moving parts.

4. The method according to claim 1, wherein said step of administering cold fluid is further defined in that said fluid is compressed, pre-cooled air stored in a tank.

5. The method according to claim 1, wherein said step of administering cold fluid is further defined in that said cold fluid is cold air, and said method comprises the further step of mixing said cold air with water to moisturize the cold air.

6. The method according to claim 1, wherein said method comprises the step of producing said cold fluid by expanding a refrigerant gas.

7. The method according to claim 1, wherein said method comprises the step of producing said cold fluid by passing air through an evaporator wherein a refrigerant is also passed through the evaporator to evaporate the refrigerant and cool the air.

8. The method according to claim 1, wherein said method comprises the step of producing said cold fluid by the use of heat transfer between the cold gas and a colder medium.

9. The method according to claim 1, wherein said method comprises the step of producing said cold fluid by introducing gas into a vortex tube.

10. The method according to claim 1, wherein said method comprises the step of producing said cold fluid by the use of heat transfer between the cold gas and a Peltier-type cooling device.

11. An apparatus to treat nosebleeds, comprising:
a source of cold gas; and
a tube in fluid communication with said source and configured to deliver said cold gas to the inside of the nose.

12. The apparatus according to claim 11, wherein said source of cold gas comprises a source of pressurized air and a vortex tube in fluid communication with said source and having a cold air output in fluid communication with said tube.

13. The apparatus according to claim 11, wherein said source of cold gas comprises a pre-cooled, pressurized air tank.

14. The apparatus according to claim 11, wherein said source of cold gas comprises a container filled with water in fluid communication with said tube, said water adding moisture to said cold gas.

15. The apparatus according to claim 11, wherein said source of cold gas comprises an air pump attached to a pressurized air tank.

16. The apparatus according to claim 11, wherein said source of cold gas comprises a container of refrigerant material.

17. The apparatus according to claim 16, wherein said refrigerant material is a compressed material which will produce cold gas upon expansion.

18. The apparatus according to claim 11, wherein said source of cold gas comprises a source of pressurized air and a refrigeration circuit having an evaporator in heat transfer with said source of pressurized air.

19. The apparatus according to claim 11, wherein said source of cold gas comprises a tube in heat transfer communication with a cold medium, said tube cooled from the outside by said cold medium as said air passes through said tube.

20. The apparatus according to claim 11, wherein said source of cold gas comprises a supply of air in heat transfer communication with a Peltier-type thermoelectric cooler.
